(19) United States
(12) Patent Application Publication
(10) Pub. No.: US 2024/0245446 A1
(43) Pub. Date: Jul. 25, 2024

(54) Multi-tool Terminus Operating Room System

(71) Applicants: Jonathan RICE, Camarillo, CA (US); Erik SPAYDE, Thousand Oaks, CA (US)

(72) Inventors: Jonathan RICE, Camarillo, CA (US); Erik SPAYDE, Thousand Oaks, CA (US)

(21) Appl. No.: 18/426,026

(22) Filed: Jan. 29, 2024

Related U.S. Application Data
(63) Continuation-in-part of application No. 18/070,440, filed on Nov. 28, 2022.
Application No. 18/070,440 is a continuation of application No. 16/877,271, filed on May 18, 2020, now Pat. No. 11,510,724.
Application No. 16/877,271 is a continuation of application No. 15/242,195, filed on Aug. 19, 2016, now Pat. No. 10,653,473.

(51) Int. Cl. A61B 18/14; A61B 18/00

(52) U.S. Cl. CPC A61B 18/14; A61B 2018/00166; A61B 2018/00178; A61B 2018/00595; A61B 2018/1475; A61B 2218/008

(57) ABSTRACT

An improved tool terminus for use in an operating room avoids the usual tangle of wires and tubes that can represent a significant hazard. The terminus in the sterile region of the operating room acts as a holder to keep the various operating instrument organized. The terminus is equipped with reels that allow the surgeon to have the optimum length of instrument lead and to retract that lead at will.

### CROSS-REFERENCE TO PRIOR APPLICATIONS

This application is a continuation-in-part of U.S. patent application Ser. No. 18/070,440, filed on Nov. 28, 2022, which was a continuation of U.S. patent application Ser. No. 16/877,271, filed May 18, 2020, now U.S. patent Ser. No. 11/510,724, which in turn was a continuation of U.S. patent application Ser. No. 15/242,195, filed Aug. 19, 2016, which issued as U.S. Pat. No. 10,653,473 on May 19, 2020.

### U.S. GOVERNMENT SUPPORT

Not applicable

### BACKGROUND OF THE INVENTION

### Area of the Art

The present invention is in the art of health care and is directed to an improved terminus system for use with commonly used surgical tools that improves the surgeon's experience, diminishes setup time, and improves sterility over the current standard.

### DESCRIPTION OF THE BACKGROUND

For much of human history the role of microorganisms in infection was unknown. It was expected that wounds either from accident or medical procedures would become inflamed and pus-filled. This was thought to be a natural part of healing. Now, of course, we know better, and great efforts are made to ensure sterility during medical procedures. However, the ever-increasing news about “super bugs” and fatal infections acquired in hospitals is resulting in increased awareness and the relevance of discovering ways to combat infections more effectively.

There appear to be several factors at play here. First, the widespread availability of antibiotics following the end of World War II has resulted in medical protocols becoming somewhat lax. Prior to the advent of antibiotics, a hospital acquired infection, particularly a surgical site infection (SSI) was serious and often fatal. As a result, great pains were taken to keep all parts of the hospital as sterile as possible. However, antibiotics were able to cure most infections so medical personnel became less and less careful. Why spend a great deal of money avoiding an infection that could easily and cheaply be cured? At the same time life-saving antibiotics became increasingly used in industrial animal husbandry to reduce cost and accelerate growth of animals raised for human consumption. Antibiotics were and are used in vast quantities to treat animal infections and generally shortcut the need for cleanliness and conditions that spread infection in industrial farms. The use of antibiotics in food production was highly profitable and seemed harmless.

However, industrial use of drugs designed to save human lives turns out to be incredibly harmful. Chronic use of antibiotics in animal husbandry causes the rapid evolution of drug-resistant “super bugs.” Many communities have become infected with drug resistant microorganisms from their drinking water. Furthermore, the meat from treated animals often contains drug resistant microorganisms because such agents are released during slaughter and contaminate the meat.

The growing prevalence of antibiotic resistance makes healthcare-associated infection increasingly serious. While some improvement has been noted, these infections remain a significant problem. The CDC Procedure Associated Module “Surgical Site Infection (SSI) Event Introduction” (January 2016 edition) states:
In 2010, an estimated 16 million operative procedures were performed in acute care hospitals in the United States. A recent prevalence study found that SSIs were the most common healthcare-associated infection, accounting for 31% of all HAIs among hospitalized patients. The CDC healthcare-associated infection (HAI) prevalence survey found that there were an estimated 157,500 surgical site infections associated with inpatient surgeries in 2011. NHSN data included 16,147 SSIs following 849,659 operative procedures in all groups reported, for an overall SSI rate of 1.9% between 2006-2008. A 19% decrease in SSI related to 10 select procedures was reported between 2008 and 2013. While advances have been made in infection control practices, including improved operating room ventilation, sterilization methods, barriers, surgical technique, and availability of antimicrobial prophylaxis, SSIs remain a substantial cause of morbidity, prolonged hospitalization, and death. SSI is associated with a mortality rate of 3%, and 75% of SSI-associated deaths are directly attributable to the SSI.

The modern operating room (OR) presents multiple dangers of infection. For many years the procedure has been to sterilize the OR as well as possible prior to use. The patient's skin is surface sterilized, and then the patient is draped with a sterile covering so that only the site of the actual operation is exposed. Instruments are sterilized and even the air is filtered to reduce or eliminate airborne contaminants. Personnel who directly approach the patient wear sterile garments and scrub prior to the operation. However, while instruments that directly contact the patient can be sterilized, a tremendous number of medical instruments (particularly electronic devices) cannot be readily sterilized. Of course, the surfaces of diagnostic devices and power supplies for instruments (e.g., electrocautery devices) can be wiped with disinfectants. However, OR sterility is further ensured by placing all such inherently non-sterile devices away from the sterile field of the OR. The wires and tubes leading from the instruments are led over or through the drape and the ends approaching the patient are sterile. Yet, as more and more complex systems are introduced, there is an increasing danger that a non-sterile tube or wire might slip or fall into the sterile region of the OR and compromise sterility. Any compromise of sterility carries the danger of an infection that may prove very difficult and very expensive to treat. Further, the large number of instruments in the OR can be difficult to manipulate optimally without a sterility imperiling slip.

### SUMMARY OF THE INVENTION

The present invention includes an improved multi-function tool terminus for use in an operating room. The inventors previously developed a multifunction cable that comes pre-sterilized and encases a plurality of wires and tubes in a protective housing making it possible to replace routing a myriad wires and tubes from one region of the operating room to another with the positioning of a single cable thus avoiding any possibility of tangling. At the sterile end of the cable the inventors created a special tool terminus that acts as a holder to keep the various operating instrument organized and easy to use. Mechanisms within the terminus allow the instruments to be individually pulled away from the terminus with the terminus unwinding additional tubing or wire as needed. When an instrument is no longer needed, a simple button push retracts the instruments into the terminus.

### DESCRIPTION OF THE FIGURES

FIG. 1 shows a diagrammatic view of a PRIOR ART operating room;

FIG. 2 is shows a diagrammatic view of an operating room with the inventive terminus in use;

FIG. 3 is a diagrammatic view of the inventive terminus from above; and

FIG. 4 shows the inventive terminus from a side view with the lid open.

### DETAILED DESCRIPTION OF THE INVENTION

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide an improved OR multi-tool terminus to help ensure OR sterility facilitating the surgeon's task by allowing ease of use, elimination of tangling of wires/tubes and seamless transitioning between use of different equipment.

With more than an estimated 234 million major surgical operations occurring worldwide every year according to the WHO (Lancet, 2008), there is a continual need to improve various aspects of surgery, whether it be to provide advantages to surgeons and assistive personnel, diminish cost, improve efficiency and/or minimize OR time. While there are variable surgical sterile and equipment setups depending on the type of surgery and surgeon preference, there are some basic drape and equipment setups that are common for most major surgical procedures. Such major operations begin with the anesthetization of a patient followed by the positioning of the patient, which is then followed by the sterile preparation of the surgical area with surgical draping and setup of surgical equipment prior to the incision or surgical approach being initiated. The most common practice implemented presently for surgical setup is the use of disposable draping with the passing of wires or tubes of surgical devices that require connection to a power source, typically located outside of the sterile field. This includes the commonly used electrocautery/electrosurgery devices (including monopolar devices known a “bovie” or “Bovie” which is a registered trademark of Bovie Medical Corporation of Clearwater, FL, bipolar forceps), suction tubing, various other fluidics and often a powered drill or saw. There can be other devices, such as light cables or other specific surgical apparatuses that may be also utilized during surgery; however uniformly, the electrocautery and suction devices with associated wiring/tubing represent the most used equipment utilized across numerous surgical practices.

The current invention is a simple, yet useful improvement involving a sterile terminus for myriad surgical tools that is cost effective, diminishes the setup time for surgical preparation and improves efficiency for surgeons/assistive personnel. The mechanics involved in the setup for commonly used surgical devices described above (monopolar instrument, bipolar forceps, and suction tubing) typically entails draping the patient sterilely and then setting up the monopolar electrosurgery device, bipolar forceps, suction tubing and drill/saw by a sterile, gowned individual who passes the source ends of the wires/tubing to an OR nurse or other non-sterile personnel, who then attach the now-contaminated end of the tubing/wiring to non-sterile power sources. The tubing and wiring are then generally secured at a certain point on the drape to allow for sufficient slack of the sterile end of the tubes and wires for the surgeon to operate and to prevent the non-sterile portion of the tubing from sliding back onto the sterile field, which would result in contamination of the sterile field.

There are numerous problems associated with this system of individually handing off numerous wires/tubes. First, each of the common non-sterile source requiring apparatuses must be uncoiled, passed to the nurse, connected to the non-sterile source, then secured to the drape, often by straps (often hook and loop fastener) that are built-in features for most disposable surgical drapes. Wires and tubes will often become tangled because a surgeon will often alternate between using different surgical tools with the ensuing tangling of tubing/wiring increasing the risk of surgical tools falling off the table or dropping low enough on the table to compromise sterility. This tangling problem can be extremely frustrating for the surgeon. A few prior art surgical devices include a station that anchors the various tubes and wires for a single tool. However, a plurality of such housings merely adds to clutter in the OR.

Rather than have separate cables which may tangle for the above-mentioned devices, a specialized, terminus is provided, which includes separate internalized connectors for bipolar forceps, monopolar electrocautery/electrosurgery, suction tubing and other apparatus such as fluidics, bipolar electrocautery with alternate embodiments of the inventive terminus incorporating additional wiring for drill and powered saw function.

The terminus arrives preassembled and pre-sterilized. Although the terminus is preferentially used with the OR cable previously disclosed by the inventors, it can also be advantageously used with individual cables traditionally employed. After the various tubes and cables are threaded into the sterile region of the OR, they are attached to the unitary terminus rather than being left as a potential tangle of separate instruments. In this way, there can be separation of wires/tubing to enable use of any of the components without interference by proximity of the other surgical instruments, while maintaining short enough lengths of the tubing/wiring on the surgeon/sterile area distal to the terminus to minimize the risk of any of the components falling off the side of the table and becoming contaminated.

The multi-tool terminus yields advantages over the primitive, existing OR standard by providing a product that:
is easier, faster, simpler to set up;
diminishes the risk of contamination of sterile field;
significantly diminishes the tangling of wiring/tubing of surgical devices, thus improving the surgeon's experience; and
reduces the incidence of surgical delays for untangling of wires/tubing or replacement of tools that have become contaminated;

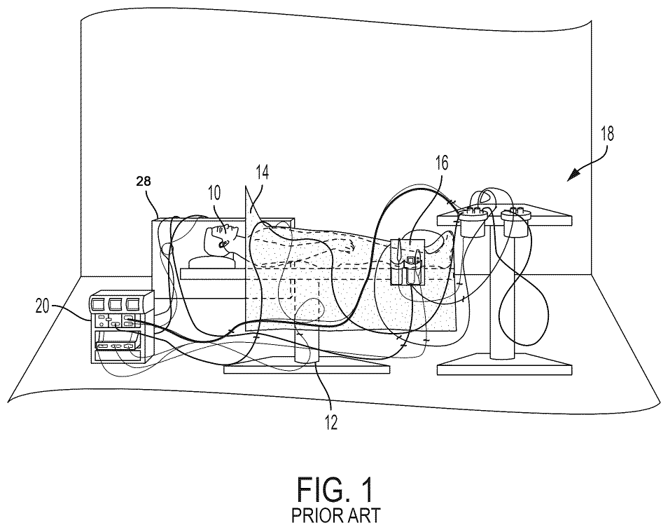

FIG. 1 shows the general layout of a PRIOR ART operating room. A patient 10 is disposed on an operating table 12 and covered with a sterile drape 14. A package of operating instruments 16 is attached to the drape 14. A veritable tangle of connecting conduits and wires connects the instruments 16 with various power sources such as suction devices 18 and bipolar cautery power supply 20. For sake of illustration, several of the power sources are shown as being very close to the patient. In actual operating rooms, these non-sterile devices may be located at a distance behind a sterile drape or barrier 28.

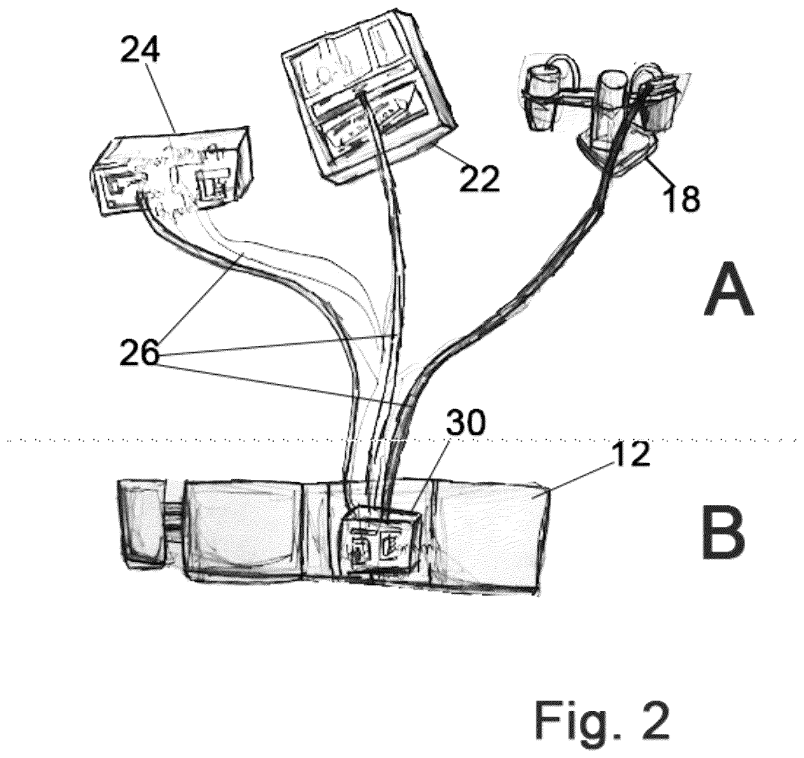

FIG. 1 should be contrasted with FIG. 2 which shows the multi-tool terminus 30 in the OR. The drawing is a diagrammatic layout to emphasize the sterile B versus non-sterile A regions of the room. In the non-sterile region A power sources for various instruments such as suction 18, bipolar electrocautery 20 and monopolar (“bovie”) electrocautery/electrosurgery 24. The A and B regions are shown in a single plane for simplicity of illustration. As previously explained in a real OR, the sterile and non-sterile regions are separated by horizontal distance along the same floor with barriers such as sterile drapes separating the regions. The various cables and tubes are threaded over a sterile drape and attached thereto. Generally, the entire floor is considered to belong to the non-sterile region.

Either the previously mentioned multifunction cable or the traditional individual cable and tubes 26 are then connected to a unitary multi-tool terminus 30. The terminus 30 can be firmly attached to the drape, bracket, shelf or table as desired (in FIG. 2 the terminus 30 appears to “float” above the OR table 12 for simplicity of illustration.) The terminus 30 contains connectors (electrical, suction and/or other fluidic sources) for the attached instruments. The terminus 30 also contains a plurality of “hose reel” retractor mechanisms 32 (see subsequent figures), one for each attachable instrument. Each reel is a spring-loaded spool around which is wound a length of tubing or wire (depending on the instrument) that connects at its distal end with a surgical instrument and at its proximal end with the proper connector within the multi-tool terminus 30.

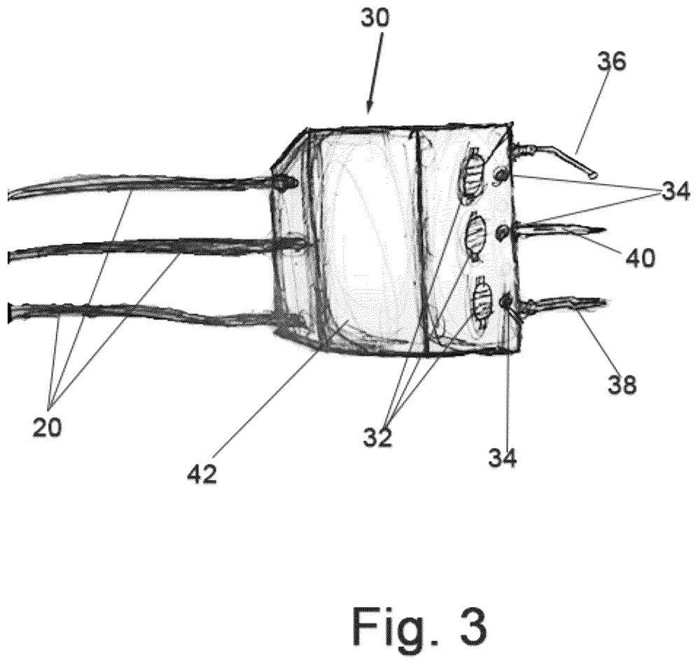

FIG. 3 shows the terminus 30 from above. The reels 32 are made visible here by rendering part of the top surface transparent. In an actual device the reels 32 would not be seen. A button 34 is associated with each instrument (suction probe 30; bipolar forceps 34; and monopolar electrosurgery instrument 38, for example). The surgeon merely pulls out an adequate length of wire or tubing for use of a given instrument during the procedure. Additional tugging will advantageously release additional wire or tubing. At any time, the surgeon can touch the appropriate button 34 on the terminus 30 to retract the instrument to the home position or to shorten the lead appropriately. In this way tangles are avoided.

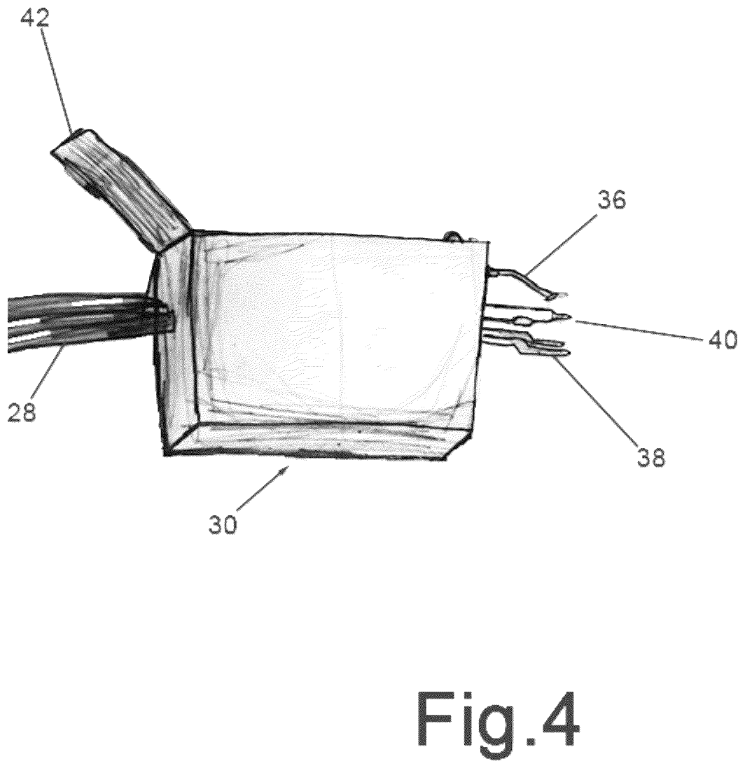

FIG. 4 shows a side view of the terminus 30 with the lid 42 open. In this embodiment of the device, the various tubes and wires 26 are inserted through an opening (not shown) in the rear panel of the terminus 30 and the lid 42 is opened to facilitate insertion of the tubes and wires 26 into appropriate connectors associated with each reel 32.

Most of the wires and conduits currently in use are disposable. Most of the operating instruments (but not the power sources) are similarly disposable although some parts, such as the monopolar electrosurgery tips, may be recycled. Although the terminus 30 could be supplied with multiple separate reel units 32 that can be inserted into the terminal 30 making it possible to “mix and match” to create a variety of different set ups, supplying the terminus 30 as a preassembled unit ensures ease of set up and minimizes contamination problems. In other words, the terminus 30 is supplied with just the reel units 32 designed for the multiple tools that will be used in a specific procedure. One goal of the invention is for use of the multi-tool terminal 30 to provide a seamless transition for surgeons and to use existing OR equipment. The current setup for monopolar electrosurgery devices, bipolar forceps and suction instruments involves passing off the wires to non-sterile personnel to attach to non-sterile sources, and selection and connection of sterile instruments of surgeon's choice to the sterile ends of the respective wires/tubes. With the multi-tool terminal 30, the sterile ends of the various tubes and wires plug into the terminus 30 which presents whatever bipolar forceps, etc. the surgeon has chosen in an easy-to-use manner. The same applies for the connection of the suction apparatus (e.g., Frasier tip, Yankauer tip).

The following claims are thus to be understood to include what is specifically illustrated and described above, what is conceptually equivalent, what can be obviously substituted. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

What is claimed is:

1. A multi-tool terminus for use in an operating room comprising: a housing disposed in a sterile region of the operating room comprising:
connectors for removably attaching multiple tools or instruments to the housing;
flexible electrical leads and flexible fluidic tubing connecting to the terminus connectors; and
a button operated retraction mechanism for releasing and retracting the flexible electrical leads and flexible fluidic tubing on demand.

2. The multi-tool terminus of claim 1, wherein said fluidic tubing comprises suction tubing.
